(19) United States
(12) Patent Application Publication
(10) Pub. No.: US 2026/0258195 A1
(43) Pub. Date: Sep. 3, 2026

(54) POLYESTER-POLYETHYLENE GLYCOL BLOCK COPOLYMER, PREPARATION METHOD THEREFOR, AND USE THEREOF

(71) Applicant: HANGZHOU SHIXI PHARMACEUTICAL TECHNOLOGY CO., LTD., Hangzhou (CN)

(72) Inventors: Ziyang Wang, Hangzhou (CN); Yongli Pei, Hangzhou (CN); Xiaoyan Wang, Hangzhou (CN); Xichen Zhang, Hangzhou (CN)

(21) Appl. No.: 19/457,234

(22) Filed: Jan. 23, 2026

(30) Foreign Application Priority Data
Feb. 28, 2025 (CN) 202510230694.6

(51) Int. Cl. C08G 63/664; C08G 63/82

(52) U.S. Cl. CPC C08G 63/664; C08G 63/823

(57) ABSTRACT

The present disclosure relates to the technical field of polymer materials, and particularly to a polyester-polyethylene glycol block copolymer, a preparation method therefor, and use thereof. The preparation method for the polyester-polyethylene glycol block copolymer includes a step of performing a melt ring-opening polymerization reaction on hydroxyl-containing polyethylene glycol and cyclic ester monomer, with stannous octoate as catalyst, where in the melt ring-opening polymerization, an amount of the stannous octoate used ranges from 0.01 wt % to 0.047 wt % of a total amount of the hydroxyl-containing polyethylene glycol and the cyclic ester monomer as raw materials; and a temperature of the melt ring-opening polymerization reaction ranges from 100° C. to 140° C., and a reaction time ranges from 48 h to 150 h.

### CROSS-REFERENCE TO RELATED APPLICATION

The present disclosure claims priority to Chinese Patent Application No. 202510230694.6 filed with the China National Intellectual Property Administration on Feb. 28, 2025 and entitled “POLYESTER-POLYETHYLENE GLYCOL BLOCK COPOLYMER, PREPARATION METHOD THEREFOR, AND USE THEREOF”, the contents of which are incorporated herein by reference in entirety.

### TECHNICAL FIELD

The present disclosure relates to the technical field of polymer materials, and particularly to a polyester-polyethylene glycol block copolymer, a preparation method therefor, and use thereof.

### BACKGROUND ART

Block copolymers of biodegradable polyesters and polyethylene glycol (including monomethyl ether thereof) (abbreviated as polyester-polyethylene glycol block copolymers) have been widely investigated and applied to drug delivery carriers and the like owing to good biocompatibility thereof. The polyesters are typically selected from homopolymers and/or copolymers of ester monomers such as lactide (LA), glycolide (GA), and caprolactone (CL). For example, diblock poly(D,L-lactide)-monomethyl ether polyethylene glycol (PDLLA-MePEG) is used to solubilize paclitaxel and docetaxel, and the resulting polymeric micelle formulation has been successfully commercialized, thus significantly reducing the side effects associated with conventional surfactant formulations.

The synthesis of polyester-polyethylene glycol block copolymers is typically carried out via ring-opening polymerization of the above ester monomers, using hydroxyl at the end of polyethylene glycol chain as an initiator and an organometallic compound as a catalyst. A specific polymerization method can be melt polymerization or solution polymerization. As an effective catalyst for ring-opening polymerization, organotin is widely employed as a catalyst in the synthesis of polyester-polyethylene glycol block copolymers. To meet the requirements of polymerization reaction and product performance, it is typically necessary to add a relatively high content of organotin catalyst. Since metal tin has certain toxicity, the content thereof in medical drugs should be lower than a certain limit. In order to comply with drug safety requirements, polymers obtained via ring-opening polymerization should undergo further purification, so as to reduce contents of residual metal, monomer, and oligomer. This reduces the production process efficiency and product yield, thus hindering the progress of widespread commercial use thereof.

In view of this, the present disclosure is specifically proposed.

### SUMMARY

The present disclosure aims at providing a polyester-polyethylene glycol block copolymer, a preparation method therefor, and a use thereof. By employing suitable polymerization reaction conditions, the polyester-polyethylene glycol block copolymer meeting requirements of drug carriers for contents of residual metal, monomer, and oligomer can be synthesized in a single step, without further purification steps.

In order to achieve the above objective of the present disclosure, in the first aspect, the present disclosure provides a preparation method for a polyester-polyethylene glycol block copolymer, comprising a step of performing a melt ring-opening polymerization reaction on hydroxyl-containing polyethylene glycol and cyclic ester monomer, with stannous octoate as catalyst.

In the melt ring-opening polymerization, an amount of the stannous octoate used ranges from 0.01 wt % to 0.047 wt % of a total amount of the hydroxyl-containing polyethylene glycol and the cyclic ester monomer as raw materials.

A temperature of the melt ring-opening polymerization reaction ranges from 100° C. to 140° C., and a reaction time ranges from 48 h to 150 h.

In embodiments of the present disclosure, the melt ring-opening polymerization reaction comprises: reacting mixed materials at 125-135° C. for 10-24 h, followed by cooling to 105-115° C., and reacting for 24-72 h.

In embodiments of the present disclosure, the stannous octoate is added directly or in the form of a premix, wherein the premix comprises:
(a) stannous octoate; and
(b) either hydroxyl-containing polyethylene glycol or cyclic ester monomer.

In embodiments of the present disclosure, in the premix, a mass fraction of the stannous octoate ranges from 0.01% to 5%.

In embodiments of the present disclosure, the melt ring-opening polymerization reaction is carried out in vacuum or under a protective gas atmosphere.

In embodiments of the present disclosure, the hydroxyl-containing polyethylene glycol comprises polyethylene glycol and monomethyl ether polyethylene glycol. Furthermore, the hydroxyl-containing polyethylene glycol has a number-average molecular weight ranging from 200 to 180,000.

In embodiments of the present disclosure, the cyclic ester monomer comprises at least one selected from the group consisting of lactide, glycolide, and caprolactone. Further, the lactide comprises at least one selected from the group consisting of meso-lactide, rac-lactide (D,L-lactide), D-lactide, and L-lactide.

In embodiments of the present disclosure, a mass ratio of the cyclic ester monomer to the hydroxyl-containing polyethylene glycol ranges from 0.4:1 to 9:1.

In the second aspect, the present disclosure provides a polyester-polyethylene glycol block copolymer, prepared by the preparation method for a polyester-polyethylene glycol block copolymer provided in the first aspect of the present disclosure.

In embodiments of the present disclosure, the polyester-polyethylene glycol block copolymer has a polydispersity Mw/Mn ≤2.0. Further, the polyester-polyethylene glycol block copolymer has a weight-average molecular weight ranging from 2,000 to 200,000.

In embodiments of the present disclosure, the polyester-polyethylene glycol block copolymer satisfies at least one of following characteristics:
(1) a metal tin content being ≤150 ppm, preferably ≤60 ppm;
(2) a residual monomer content being ≤3.0 wt %, preferably ≤2.0 wt %; and
(3) an oligomer content being ≤3.0 wt %, preferably ≤2.0 wt %.

In the third aspect, the present disclosure provides a use of the polyester-polyethylene glycol block copolymer provided in the second aspect of the present disclosure in the manufacture of a drug carrier.

In embodiments of the present disclosure, the drug comprises at least one selected from the group consisting of nimodipine, paclitaxel, docetaxel, indomethacin, and cinacalcet hydrochloride.

Compared with the prior art, the present disclosure has following beneficial effects.

(1) By employing suitable polymerization reaction conditions, the present disclosure synthesizes the polyester-polyethylene glycol block copolymer meeting requirements of drug carriers for contents of residual metal, monomer, and oligomer in a single step, without purification steps, thus shortening the process flow, improving the production efficiency and product yield, and facilitating wider commercial use.

(2) The polyester-polyethylene glycol block copolymer prepared by the preparation method of the present disclosure has metal tin residue and monomer residue that conform to the national standards for PLA and PLGA implantable materials, and the metal tin content conforms to the standard requirements (oral administration ≤600 ppm and injection ≤60 ppm) of option 1 (Table A.2.2) in ICH Q3D (R1).

(3) The polyester-polyethylene glycol block copolymer of the present disclosure can meet the requirements for use as a drug carrier, and can be directly employed for manufacturing formulations such as drug-loaded polymer micelles.

### BRIEF DESCRIPTION OF DRAWINGS

In order to more clearly illustrate technical solutions in embodiments of the present disclosure or the prior art, drawings that need to be used in the description of the embodiments or the prior art will be briefly introduced below. Obviously, the drawings in the following description show some embodiments of the present disclosure, and those ordinarily skilled in the art still could obtain other drawings according to these drawings without using any inventive efforts.

FIG. 1 is an HPLC detection chromatogram of a polyester-polyethylene glycol block copolymer prepared in Example 13 of the present disclosure;

FIG. 2 is a comparison diagram of lactide residue amounts in polyester-polyethylene glycol determined by the HPLC detection method and the ¹H-NMR detection method of the present disclosure;

FIG. 3 is a GPC chromatogram of the polyester-polyethylene glycol block copolymer prepared in Example 13 of the present disclosure;

FIG. 4 shows influence of catalyst dosage and reaction time on lactide residue amount in the polyester-polyethylene glycol block copolymer at 130° C. provided in the present disclosure;

FIG. 5 shows influence of catalyst dosage and reaction time on lactide residue amount in the polyester-polyethylene glycol block copolymer at 140° C. provided in the present disclosure;

FIG. 6 shows influence of catalyst dosage and reaction time on oligomer amount in the polyester-polyethylene glycol block copolymer at 130° C. provided in the present disclosure; and

FIG. 7 shows influence of catalyst dosage and reaction time on oligomer amount in the polyester-polyethylene glycol block copolymer at 140° C. provided in the present disclosure.

### DETAILED DESCRIPTION OF EMBODIMENTS

Technical solutions of the present disclosure will be described clearly and completely below in conjunction with the drawings and embodiments, while those skilled in the art would understand that the examples described below are some but not all examples of the present disclosure, and they are merely used for illustrating the present disclosure, but should not be considered as limiting the scope of the present disclosure. Based on the embodiments in the present disclosure, all of other embodiments obtained by those ordinarily skilled in the art without using any inventive efforts shall fall within the scope of protection of the present disclosure. Examples, for which no concrete conditions are specified, are carried out according to conventional conditions or conditions recommended by manufactures. If manufacturers of reagents or apparatuses used are not specified, all of them are conventional products commercially available

Existing synthesis of polyester-polyethylene glycol block copolymers is typically carried out via melt polymerization or solution polymerization. Herein, the solution polymerization process is relatively simple, but requires use of a solvent, which, on the one hand, compromises the environmental protection, and on the other hand, increases production costs due to use of a large amount of solvent, moreover, a removal process of the solvent after the polymerization not only reduces the production efficiency, but also increases the process costs. In the melt polymerization process, organotin such as stannous octoate is mostly used as a catalyst. To ensure the progress of polymerization reaction and the performance of product, stannous octoate is typically added at 0.3 wt % (equivalent to a tin content of 879 ppm) of a total reactant mass or higher. According to the ICH Q3D guidelines, and the national standards YY/T 0510-2009 and YY/T 0661-2017 for PLA and PLGA implantable materials, in order to comply with drug safety requirements, the polymer prepared in the presence of 0.3 wt % of catalyst should undergo purification steps, including but not limited to dissolution, adsorption, precipitation, and drying, to reduce contents of residual metals and monomers, which leads to reduction in production process efficiency and product yield. Moreover, materials employed in the ring-opening polymerization are costly. Multiple factors hinder wider commercial use of such polymer products.

On this basis, in the first aspect, the present disclosure provides a preparation method for a polyester-polyethylene glycol block copolymer, including a step of performing a melt ring-opening polymerization reaction on hydroxyl-containing polyethylene glycol and cyclic ester monomer, with stannous octoate as catalyst.

In the melt ring-opening polymerization, an amount of stannous octoate used ranges from 0.01 wt % to 0.047 wt % of a total amount of the hydroxyl-containing polyethylene glycol and the cyclic ester monomer as raw materials.

A temperature of the melt ring-opening polymerization reaction ranges from 100° C. to 140° C., and a reaction time ranges from 48 h to 150 h.

In the preparation of the block copolymer of the present disclosure, by employing stannous octoate as catalyst, reducing the addition amount of stannous octoate, and in combination with specific polymerization reaction temperature and reaction time, the prepared block copolymer, without purification, exhibits monomer residue ≤3.0 wt %, oligomer content ≤3.0 wt %, and metal tin residue ≤150 ppm, complying with the national standards for PLA and PLGA implantable materials, and the metal tin content conforms to the standard requirements of option 1 (Table A.2.2) in ICH Q3D (R1).

For example, in different embodiments, the amount of stannous octoate used can be 0.01 wt %, 0.015 wt %, 0.02 wt %, 0.025 wt %, 0.03 wt %, 0.035 wt %, 0.04 wt %, 0.045 wt %, or 0.047 wt % of the total amount of the hydroxyl-containing polyethylene glycol and the cyclic ester monomer as raw materials, or in a range constituted by any two thereof. Thus, while ensuring that the ring-opening polymerization proceeds smoothly and the molecular weight of the block copolymer and the contents of residual monomer and oligomer meet the requirements of the preparation, the addition amount of stannous octoate is significantly reduced, and the requirements on metal tin content in oral preparations or even injection preparations can be met without purification treatment. When the amount of stannous octoate used is less than the above range, it is difficult to meet the requirements such as conversion rate, monomer residue, and copolymer molecular weight.

For example, in different embodiments, the temperature of the melt ring-opening polymerization reaction can be 100° C., 105° C., 110° C., 115° C., 120° C., 125° C., 130° C., 135° C., or 140° C., or in a range constituted by any two thereof; the reaction time can be 48 h, 60 h, 72 h, 96 h, 120 h, 140 h, or 150 h, or in a range constituted by any two thereof. When the above temperature is lower than a melting point of the cyclic ester monomer, in actual operations, the preparation method includes: heating the hydroxyl-containing polyethylene glycol to above the melting point of the cyclic ester monomer in advance (such as 5-30° C. above the melting point), then adding the cyclic ester monomer, and upon melting of the cyclic ester monomer, cooling to a corresponding melt ring-opening polymerization temperature to perform the melt ring-opening polymerization reaction. It is thereby more conducive to making the color of the block copolymer lighter.

In embodiments of the present disclosure, the preparation method for the polyester-polyethylene glycol block copolymer excludes purification steps. Herein, the purification steps include, but are not limited to, dissolution, adsorption, precipitation, drying, etc.

In embodiments of the present disclosure, the melt ring-opening polymerization reaction includes: reacting mixed materials at 125-135° C. for 10-24 h, followed by cooling to 105-115° C., and reacting for 24-72 h.

The inventors of the present disclosure inventively found that, in the melt ring-opening polymerization reaction, by employing a low addition amount of stannous octoate, further regulating the reaction temperature in coordination, reacting at a relatively high temperature for a period of time, and then appropriately reducing the temperature and reacting for a period of time, the contents of residual monomer and oligomer can be reduced, and the color of the block copolymer can be lighter while being capable of obtaining the block copolymer with a desired molecular weight. For example, the mixed materials can be first reacted at 125° C., 128° C., 130° C., 132° C., or 135° C., or in a range constituted by any two thereof for 10 h, 12 h, 15 h, 18 h, 20 h, or 24 h, or in a range constituted by any two thereof, and then cooled to 105° C., 108° C., 110° C., 111° C., 112° C., 113° C., 114° C., or 115° C., or in a range constituted by any two thereof, followed by further reaction for 24 h, 30 h, 36 h, 40 h, 48 h, 60 h, or 72 h, or in a range constituted by any two thereof. Herein, the mixed materials refer to a mixture of hydroxyl-containing polyethylene glycol, cyclic ester monomer, and stannous octoate.

In embodiments of the present disclosure, stannous octoate is added directly or in the form of a premix, wherein the premix comprises:
(a) stannous octoate; and
(b) either hydroxyl-containing polyethylene glycol or cyclic ester monomer.

In the present disclosure, by introducing stannous octoate in the form of a premix into a polymerization reaction system, stannous octoate is dosed more accurately, which is more conducive to improving the mixing uniformity of stannous octoate in the mixing reaction system, improving the reaction uniformity, and mitigating the risk of too broad molecular weight distribution caused by non-uniform reaction.

In an embodiment of the present disclosure, the premix can comprise stannous octoate and hydroxyl-containing polyethylene glycol. The preparation of the premix includes, but is not limited to: compounding and mixing proportionally, heating to melt the hydroxyl-containing polyethylene glycol, and mixing uniformly to yield the premix, wherein a temperature of the heating can be 70-90° C., such as 70° C., 75° C., 80° C., 85° C., or 90° C.

In another embodiment of the present disclosure, the premix can comprise stannous octoate and cyclic ester monomer, and the preparation of the premix includes, but is not limited to: uniformly mixing a cyclohexane solution of stannous octoate with the cyclic ester monomer proportionally, and then performing vacuum evacuation to remove cyclohexane, yielding the premix, wherein a concentration of stannous octoate in the cyclohexane solution of stannous octoate can be 10-120 mg/mL, such as 10 mg/mL, 40 mg/mL, 50 mg/mL, 60 mg/mL, 70 mg/mL, 80 mg/mL, 90 mg/mL, 100 mg/mL, 110 mg/mL, or 120 mg/mL.

In embodiments of the present disclosure, in the premix, a mass fraction of stannous octoate is 0.01%-5%, for example, it can be 0.01%, 0.1%, 0.5%, 1%, 2%, 3%, 4%, or 5%, or in a range constituted by any two thereof, thus being more conducive to ensuring uniformity of stannous octoate mixing, and improving the molecular weight distribution width of the block copolymer while ensuring the polymerization reaction efficiency.

In embodiments of the present disclosure, the melt ring-opening polymerization reaction is carried out in vacuum or under a protective gas atmosphere. Herein, the protective gas atmosphere includes at least one of nitrogen and argon.

In embodiments of the present disclosure, the cyclic ester monomer is added to the molten hydroxyl-containing polyethylene glycol, and after uniform mixing, stannous octoate is added, followed by rapid uniform mixing, and performing the melt ring-opening polymerization reaction.

In embodiments of the present disclosure, the method further includes: performing a pretreatment on the hydroxyl-containing polyethylene glycol, wherein the pretreatment includes: performing vacuum evacuation on the hydroxyl-containing polyethylene glycol in a molten state to remove moisture. Herein, in the pretreatment, the hydroxyl-containing polyethylene glycol can be heated to 110-130° C. to melt the hydroxyl-containing polyethylene glycol, and maintained in this state for performing the vacuum evacuation.

In embodiments of the present disclosure, the hydroxyl-containing polyethylene glycol comprises polyethylene glycol and monomethyl ether polyethylene glycol. Furthermore, the hydroxyl-containing polyethylene glycol has a number-average molecular weight ranging from 200 to 180,000, preferably ranging from 2,000 to 8,000.

A hydroxyl-containing polyethylene glycol raw material in the present disclosure can be selected from raw materials conforming to the pharmacopeia standards or equivalents. The hydroxyl-containing polyethylene glycol in the present disclosure can be any one or more selected from the group consisting of linear polyethylene glycol, branched polyethylene glycol, and star-shaped polyethylene glycol.

In embodiments of the present disclosure, the cyclic ester monomer comprises at least one selected from the group consisting of lactide, glycolide (GA), and caprolactone (ε-CL). Further, lactide comprises at least one selected from the group consisting of meso-lactide (meso-LA), rac-lactide (DLLA), D-lactide (DLA), and L-lactide (LLA). Purity of the cyclic ester monomer raw material in the present disclosure is ≥99 wt %.

In embodiments of the present disclosure, a mass ratio of the cyclic ester monomer to the hydroxyl-containing polyethylene glycol ranges from 0.4:1 to 9:1, for example, it can be 0.4:1, 0.5:1, 0.6:1, 0.7:1, 0.8:1, 0.9:1, 1:1, 3:1, 5:1, or 9:1, or in a range constituted by any two thereof, thereby being more beneficial to satisfy usage requirements of polymer micelle formulations.

In the second aspect, the present disclosure provides a polyester-polyethylene glycol block copolymer, prepared by the preparation method for the polyester-polyethylene glycol block copolymer provided in the first aspect of the present disclosure.

In embodiments of the present disclosure, the polyester-polyethylene glycol block copolymer has a polydispersity Mw/Mn ≤2.0, preferably ≤1.2. Furthermore, the polyester-polyethylene glycol block copolymer has a weight-average molecular weight ranging from 2,000 to 200,000.

The polydispersity of the polyester-polyethylene glycol block copolymer of the present disclosure is a ratio of the weight-average molecular weight Mw to the number-average molecular weight Mn of the polyester-polyethylene glycol block copolymer as determined by GPC. The polyester-polyethylene glycol block copolymer prepared by the preparation method of the present disclosure not only has the desired molecular weight, but also has relatively narrow molecular weight distribution width, which is more beneficial to improve the stability and the like of the polyester-polyethylene glycol block copolymer as a pharmaceutical carrier formulation. For example, in different embodiments, the polyester-polyethylene glycol block copolymer can have the polydispersity Mw/Mn of 2.0, 1.6, 1.2, 1.19, 1.18, 1.16, 1.15, 1.14, 1.12, 1.1, 1.08, or 1.06, or in a range constituted by any two thereof, and the polyester-polyethylene glycol block copolymer can have the weight-average molecular weight of 2,000, 4,000, 5,000, 6,000, 7,000, 8,000, 9,000, 10,000, 50,000, 100,000, 150,000, or 200,000 or in a range constituted by any two thereof, thus satisfying the formulation requirements.

In the present disclosure, when the polyester-polyethylene glycol block copolymer is measured by GPC, in a specific GPC method, a chromatographic column is GPC KF-803L (8.0 mm×300 mm, 6 μm), purchased from Resonac Holdings Corporation; tetrahydrofuran is employed as a mobile phase, isocratic elution is performed at a flow rate of 1.0 mL/min, a column temperature is 40° C., a sample injection amount is 20 μL, the detector is AD differential refractive index detector, and an analysis duration is 15 min. The standard in the GPC assay is polyethylene glycol.

In embodiments of the present disclosure, the polyester-polyethylene glycol block copolymer satisfies at least one of following characteristics:
(1) a metal tin content being ≤150 ppm, preferably ≤60 ppm, wherein for example, the metal tin content can be 150 ppm, 120 ppm, 100 ppm, 90 ppm, 80 ppm, 60 ppm, or 50 ppm, or in a range constituted by any two thereof;
(2) a residual monomer content being ≤3.0 wt %, preferably ≤2.0 wt %, wherein for example, the residual monomer content can be 3.0 wt %, 2.8 wt %, 2.5 wt %, 2.2 wt %, 2 wt %, 1.8 wt %, 1.5 wt %, 1.2 wt %, or 1 wt %, or in a range constituted by any two thereof; and
(3) an oligomer content being ≤3.0 wt %, preferably ≤2.0 wt %, wherein for example, the oligomer content can be 3.0 wt %, 2.5 wt %, 2.2 wt %, 2 wt %, 1.8 wt %, 1.5 wt %, 1.2 wt %, 1 wt %, or 0.9 wt %, or in a range constituted by any two thereof. Herein, oligomer in the present disclosure refers to low-molecular-weight polymers (oligomers) with a molecular weight ranging from 162 to 1026.

In the third aspect, the present disclosure provides a use of the polyester-polyethylene glycol block copolymer provided in the second aspect of the present disclosure in the manufacture of a drug carrier.

In embodiments of the present disclosure, the drug includes at least one selected from the group consisting of nimodipine, paclitaxel, docetaxel, indomethacin, and cinacalcet hydrochloride.

The present disclosure further provides a method for detecting a monomer and/or oligomer content in a polyester-polyethylene glycol block copolymer, including a step of performing high performance liquid chromatography detection on a test solution.

Conditions for the high performance liquid chromatography detection include:
a chromatographic column being a C18 chromatographic column; a column temperature being 40° C.; and a detection wavelength being 210 nm;
performing gradient elution using a mobile phase A and a mobile phase B, at a flow rate of 1.0 mL/min, where the mobile phase A is an aqueous phosphoric acid solution with a mass fraction of 0.1%, and the mobile phase B is acetonitrile; and
a gradient elution process including: 0-2 min, a volume ratio of the mobile phase A to the mobile phase B being 98:2; 2-25 min, changing the volume ratio of the mobile phase A to the mobile phase B from 98:2 to 0:100; 25-30 min, the volume ratio of the mobile phase A to the mobile phase B being 0:100; within 30-31 min, changing the volume ratio of the mobile phase A to the mobile phase B from 0:100 to 98:2; and 31-41 min, the volume ratio of the mobile phase A to the mobile phase B being 98:2.

Herein, the monomer content in test sample can be calculated by an external standard method. Specifically, a calculation method for the monomer content includes: injecting a series of standard working solutions of the monomer (such as lactide) into high performance liquid chromatography instrument, respectively, measuring corresponding peak areas under the same chromatographic conditions as those for the test solution, and plotting a standard curve with the concentration of the monomer in the series of standard working solutions as the abscissa, and the peak area as the ordinate; obtaining a chromatographic peak area corresponding to the monomer in the test solution according to the chromatogram of the test solution, and substituting the same into the standard curve to calculate the concentration of the monomer in the test solution. Herein, the series of standard working solutions can be acetonitrile solutions of the monomer with the monomer concentration ranging from 1 mg/mL to 10 mg/mL.

In embodiments of the present disclosure, the preparation of the test solution includes: dissolving the polyester-polyethylene glycol block copolymer to be tested in acetonitrile, followed by filtration to yield a filtrate. Further, in the test solution, the concentration of the polyester-polyethylene glycol block copolymer to be tested ranges from 25 ng/ml to 35 ng/ml, for example, 25 mg/mL, 28 mg/mL, 30 mg/mL, 32 mg/mL, or 35 mg/mL.

In embodiments of the present disclosure, the chromatographic column is Supersil AQ-C18, 5 μm, 4.6 mm×250 mm.

In embodiments of the present disclosure, the sample injection amount in the high performance liquid chromatography detection can be 10 μL.

By the detection method of the present disclosure, the monomer residue and oligomer contents in the polymer can be quickly and accurately determined, facilitating improving the synthesis efficiency and controlling the polymer quality. The monomer residue amount determined by the detection method of the present disclosure is highly consistent with that through the conventional ¹H-NMR measurement.

Corresponding meanings or full names of some English abbreviations involved in the present disclosure are listed below:
DLLA: D,L-lactide;
mLA: meso-lactide;
LLA: L-lactide;
DLA: D-lactide;
CL: ε-caprolactone;
GA: glycolide;
MePEG: methoxy polyethylene glycol;
PEG: polyethylene glycol;
PLLA: poly(L-lactide);
PDLA: poly(D-lactide);
PDLLA/LLA: poly(D,L-lactide-co-L-lactide);
PDLLA/DLA: poly(D,L-lactide-co-D-lactide);
PDLLA/LLA-PEG-PDLLA/LLA: poly(D,L-lactide-co-L-lactide)-block-polyethylene glycol-block-poly(D,L-lactide-co-L-lactide);
in PDLLA/LLA-PEG-PDLLA/LLA W-X/Y/Z in the subsequent text, W represents the molecular weight of PEG, X, Y, and Z respectively represent the mass fractions (wt %) of D,L-lactide, L-lactide, and PEG in the polymer; and
in PLLA-PEG-PLLA W-Y/Z in the subsequent text, W represents the molecular weight of PEG, and Y and Z represent the mass fractions (wt %) of L-lactide and PEG in the polymer, respectively.

### Example 1

The present example provides a preparation method for a polyester-polyethylene glycol block copolymer, including following steps.

To a 100 mL clean flask equipped with a magnetic stirrer, 30 g of PEG with the molecular weight of 3350 was added. The flask was immersed in the oil bath at 110° C. Upon PEG melting, vacuum was applied for about 16 h under stirring to remove volatile substances such as residual moisture from the PEG. Upon heating to 130° C., 10 g of D,L-lactide and 10 g of L-lactide were added, respectively. After melting, 0.018 g of stannous octoate was added. The polymerization reaction was carried out with stirring at 130° C. for 96 h, yielding the block copolymer PDLLA/LLA-PEG-PDLLA/LLA 3350-20/20/60. In the above, after each addition of materials, brief evacuation, for example, for 30 min, was applied, followed by sealing.

### Example 2

The present example provides a preparation method for a polyester-polyethylene glycol block copolymer, including following steps.

To a 100 mL clean flask equipped with a magnetic stirrer, 30 g of PEG with the molecular weight of 3350 was added. The flask was immersed in the oil bath at 110° C. Upon PEG melting, vacuum was applied for about 16 h under stirring to remove volatile substances such as residual moisture from the PEG. Upon heating to 130° C., 10 g of D,L-lactide and 10 g of L-lactide were added, respectively. After complete melting, temperature was reduced to 110° C., and 0.0209 g of stannous octoate was added. The polymerization reaction was carried out with stirring at 110° C. for 150 h, yielding the block copolymer PDLLA/LLA-PEG-PDLLA/LLA 3350-20/20/60. In the above, after each addition of materials, brief evacuation, for example, for 30 min, was applied, followed by sealing.

### Example 3

The present example provides a preparation method for a polyester-polyethylene glycol block copolymer, including following steps.

To a 100 mL clean flask equipped with a magnetic stirrer, 30 g of PEG with the molecular weight of 3350 was added. The flask was immersed in the oil bath at 110° C. Upon PEG melting, vacuum was applied for about 16 h under stirring to remove volatile substances such as residual moisture from the PEG. Upon heating to 130° C., 10 g of D,L-lactide and 10 g of L-lactide were added, respectively. After complete melting, temperature was reduced to 110° C., and 0.0235 g of stannous octoate was added. The polymerization reaction was carried out with stirring at 100° C. for 150 h, yielding the block copolymer PDLLA/LLA-PEG-PDLLA/LLA 3350-20/20/60. In the above, after each addition of materials, brief evacuation, for example, for 30 min, was applied, followed by sealing.

### Example 4

The present example provides a preparation method for a polyester-polyethylene glycol block copolymer, including following steps.

To a 100 mL clean flask equipped with a magnetic stirrer, 30 g of PEG with the molecular weight of 3350 was added. The flask was immersed in the oil bath at 110° C. Upon PEG melting, vacuum was applied for about 16 h under stirring to remove volatile substances such as residual moisture from the PEG. Upon heating to 140° C., stannous octoate/lactide mixture was added. The polymerization reaction was carried out with stirring at 140° C. for 48 h, yielding the block copolymer PDLLA/LLA-PEG-PDLLA/LLA 3350-20/20/60. In the above, after each addition of materials, brief evacuation, for example, for 30 min, was applied, followed by sealing.

Herein, the preparation of the stannous octoate/lactide mixture included: taking 0.16 mL of cyclohexane solution of stannous octoate with concentration of 50 mg/mL, adding 10 g of D,L-lactide and 10 g of L-lactide, uniformly mixing, and then performing vacuum evacuation to remove cyclohexane. The entire resultant was added to the above reaction system.

### Example 5

The present example provides a preparation method for a polyester-polyethylene glycol block copolymer, including following steps.

To a 100 mL clean flask equipped with a magnetic stirrer, 30 g of PEG with the molecular weight of 3350 was added. The flask was immersed in the oil bath at 110° C. Upon PEG melting, vacuum was applied for about 16 h under stirring to remove volatile substances such as residual moisture from the PEG. Upon heating to 140° C., 10 g of D,L-lactide and 10 g of L-lactide were added, respectively. After melting, 0.016 g of stannous octoate was added. The polymerization reaction was carried out with stirring at 140° C. for 96 h, yielding the block copolymer PDLLA/LLA-PEG-PDLLA/LLA 3350-20/20/60. In the above, after each addition of materials, brief evacuation, for example, for 30 min, was applied, followed by sealing.

### Example 6

The present example provides a preparation method for a polyester-polyethylene glycol block copolymer, including following steps.

To a 10 mL clean flask equipped with a magnetic stirrer, 5 g of PEG with the molecular weight of 6000 was added. The flask was immersed in the oil bath at 130° C. Upon PEG melting, vacuum was applied for about 2 h under stirring to remove volatile substances such as residual moisture from the PEG. 5 g of L-lactide was added. After melting, 0.0047 g of stannous octoate was added. The polymerization reaction was carried out with stirring at 130° C. for 48 h, yielding the block copolymer PLLA-PEG-PLLA 6000-50/50. In the above, after each addition of materials, brief evacuation, for example, for 30 min, was applied, followed by sealing.

### Example 7

The present example provides a preparation method for a polyester-polyethylene glycol block copolymer, including following steps.

To a 10 mL clean flask equipped with a magnetic stirrer, 5 g of PEG with the molecular weight of 6000 was added. The flask was immersed in the oil bath at 130° C. Upon PEG melting, vacuum was applied for about 2 h under stirring to remove volatile substances such as residual moisture from the PEG. 5 g of L-lactide was added. After melting, 0.0029 g of stannous octoate was added. The polymerization reaction was carried out with stirring at 130° C. for 72 h, yielding the block copolymer PLLA-PEG-PLLA 6000-50/50. In the above, after each addition of materials, brief evacuation, for example, for 30 min, was applied, followed by sealing.

### Example 8

The present example provides a preparation method for a polyester-polyethylene glycol block copolymer, including following steps.

To a 100 mL clean flask equipped with a magnetic stirrer, 30 g of PEG with the molecular weight of 3350 was added. The flask was immersed in the oil bath at 110° C. Upon PEG melting, vacuum was applied for about 16 h under stirring to remove volatile substances such as residual moisture from the PEG. Upon heating to 130° C., 10 g of D,L-lactide and 10 g of L-lactide were added, respectively. After melting, 0.11 g of stannous octoate/PEG 3350 mixture was added, followed by stirring at 130° C. for 72 h. 0.056 g of stannous octoate/PEG 3350 mixture was supplemented, followed by further stirring at 130° C. until reacting for 150 h, thereby completing the polymerization reaction, and yielding the block copolymer PDLLA/LLA-PEG-PDLLA/LLA 3350-20/20/60. In the above, after each addition of materials, brief evacuation, for example, for 30 min, was applied, followed by sealing.

Herein, the preparation of the stannous octoate/PEG 3350 mixture included: weighing stannous octoate and PEG with the molecular weight of 3350 in a mass ratio of 1:19, melting the PEG at 80° C., uniformly mixing the melted PEG with stannous octoate, and cooling to obtain the stannous octoate/PEG 3350 mixture.

### Example 9

The present example provides a preparation method for a polyester-polyethylene glycol block copolymer, including following steps.

To a 10 mL clean flask equipped with a magnetic stirrer, 4.8 g of PEG with the molecular weight of 6000 was added. The flask was immersed in the oil bath at 130° C. Upon PEG melting, vacuum was applied for about 2 h under stirring to remove volatile substances such as residual moisture from the PEG. 5 g of L-lactide was added. After melting, 0.16 g of stannous octoate/PEG 6000 mixture was added, followed by stirring at 130° C. for 48 h, thereby completing the polymerization reaction, and yielding the block copolymer PLLA-PEG-PLLA 6000-50/50. In the above, after each addition of materials, brief evacuation, for example, for 30 min, was applied, followed by sealing.

Herein, the preparation of the stannous octoate/PEG 6000 mixture included: weighing stannous octoate and PEG with the molecular weight of 6000 in a mass ratio of 1:39, melting the PEG at 80° C., uniformly mixing the melted PEG with stannous octoate, and cooling to obtain the stannous octoate/PEG 6000 mixture.

### Example 10

The present example provides a preparation method for a polyester-polyethylene glycol block copolymer, including following steps.

To a 10 mL clean flask equipped with a magnetic stirrer, 4.95 g of PEG with the molecular weight of 6000 was added. The flask was immersed in the oil bath at 130° C. Upon PEG melting, vacuum was applied for about 2 h under stirring to remove volatile substances such as residual moisture from the PEG. 5 g of L-lactide was added. After melting, 0.065 g of stannous octoate/PEG 6000 mixture was added, followed by stirring at 130° C. for 72 h, thereby completing the polymerization reaction, and yielding the block copolymer PLLA-PEG-PLLA 6000-50/50. In the above, after each addition of materials, brief evacuation, for example, for 30 min, was applied, followed by sealing.

Herein, the preparation of the stannous octoate/PEG 6000 mixture followed Example 9.

### Example 11

The present example provides a preparation method for a polyester-polyethylene glycol block copolymer, including following steps.

To a 100 mL clean flask equipped with a magnetic stirrer, 30 g of PEG with the molecular weight of 3350 was added. The flask was immersed in the oil bath at 110° C. Upon PEG melting, vacuum was applied for about 16 h under stirring to remove volatile substances such as residual moisture from the PEG. Upon heating to 130° C., 10 g of D,L-lactide and 10 g of L-lactide were added, respectively. After melting, 0.019 g of stannous octoate was added, followed by stirring at 130° C. for 24 h, then cooling to 110° C., and further stirring for 48 h, thereby completing the polymerization reaction, and yielding the block copolymer PDLLA/LLA-PEG-PDLLA/LLA 3350-20/20/60. In the above, after each addition of materials, brief evacuation, for example, for 30 min, was applied, followed by sealing.

### Example 12

The present example provides a preparation method for a polyester-polyethylene glycol block copolymer, including following steps.

To a 250 mL clean flask equipped with a magnetic stirrer, 30 g of PEG with the molecular weight of 3350 was added. The flask was immersed in the oil bath at 110° C. Upon PEG melting, vacuum was applied for about 16 h under stirring to remove volatile substances such as residual moisture from the PEG. Upon heating to 130° C., 10 g of D,L-lactide and 10 g of L-lactide were added, respectively. After melting, 0.16 g of stannous octoate/PEG 3350 mixture was added, followed by stirring at 130° C. for 24 h, then cooling to 110° C., and further stirring for 48 h, thereby completing the polymerization reaction, and yielding the block copolymer PDLLA/LLA-PEG-PDLLA/LLA 3350-20/20/60. In the above, after each addition of materials, brief evacuation, for example, for 30 min, was applied, followed by sealing.

Herein, the preparation of the stannous octoate/PEG 3350 mixture followed Example 8.

### Example 13

The present example provides a preparation method for a polyester-polyethylene glycol block copolymer, including following steps.

To a 1000 mL clean flask equipped with a magnetic stirrer, 570 g of PEG with the molecular weight of 6000 was added. The flask was immersed in the oil bath at 130° C. Upon PEG melting, vacuum was applied for about 17 h under stirring to remove volatile substances such as residual moisture from the PEG. 400 g of L-lactide was added. After melting, 30 g of stannous octoate/L-lactide mixture was added, followed by stirring at 130° C. for 24 h, then cooling to 110° C., and further stirring for 48 h, thereby completing the polymerization reaction, and yielding the block copolymer PLLA-PEG-PLLA 6000-43/57. In the above, after each addition of materials, brief evacuation, for example, for 60 min, was applied, followed by sealing.

Herein, the preparation of the stannous octoate/L-lactide mixture included: taking 3 mL of cyclohexane solution of stannous octoate with concentration of 100 mg/mL, adding 30 g of L-lactide, uniformly mixing, and then performing vacuum evacuation to remove cyclohexane.

### Example 14

The present example provides a preparation method for a polyester-polyethylene glycol block copolymer, including following steps.

To a 1000 mL clean flask equipped with a magnetic stirrer, 570 g of PEG with the molecular weight of 6000 was added. The flask was immersed in the oil bath at 130° C. Upon PEG melting, vacuum was applied for about 17 h under stirring to remove volatile substances such as residual moisture from the PEG. 430 g of D-lactide was added. After melting, 0.32 g of stannous octoate was added, followed by stirring at 130° C. for 24 h, then cooling to 110° C., and further stirring for 48 h, thereby completing the polymerization reaction, and yielding the block copolymer PDLA-PEG-PDLA 6000-43/57. In the above, after each addition of materials, brief evacuation, for example, for 60 min, was applied, followed by sealing.

### Example 15

The present example provides a preparation method for a polyester-polyethylene glycol block copolymer, including following steps.

To a 10 mL clean flask equipped with a magnetic stirrer, 5.7 g of PEG with the molecular weight of 6000 was added. The flask was immersed in the oil bath at 130° C. Upon PEG melting, vacuum was applied for about 1.5 h under stirring to remove volatile substances such as residual moisture from the PEG. 1.43 g of D,L-lactide and 2.57 g L-lactide were added, respectively. After melting, 0.3 g of stannous octoate/L-lactide mixture was added, followed by stirring at 130° C. for 24 h, then cooling to 110° C., and further stirring for 48 h, thereby completing the polymerization reaction, and yielding the block copolymer PDLLA/LLA-PEG-PDLLA/LLA 6000-14.3/28.7/57. In the above, after each addition of materials, brief evacuation, for example, for 30 min, was applied, followed by sealing.

Herein, the preparation of the stannous octoate/L-lactide mixture followed Example 13.

### Example 16

The present example provides a preparation method for a polyester-polyethylene glycol block copolymer, including following steps.

To a 10 mL clean flask equipped with a magnetic stirrer, 5.7 g of PEG with the molecular weight of 6000 was added. The flask was immersed in the oil bath at 130° C. Upon PEG melting, vacuum was applied for about 1.5 h under stirring to remove volatile substances such as residual moisture from the PEG. 0.86 g of D,L-lactide and 3.14 g of L-lactide were added, respectively. After melting, 0.3 g of stannous octoate/L-lactide mixture was added, followed by stirring at 130° C. for 24 h, then cooling to 110° C., and further stirring for 48 h, thereby completing the polymerization reaction, and yielding the block copolymer PDLLA/LLA-PEG-PDLLA/LLA 6000-8.6/34.4/57. In the above, after each addition of materials, brief evacuation, for example, for 30 min, was applied, followed by sealing.

Herein, the preparation of the stannous octoate/L-lactide mixture followed Example 13.

### Example 17

The present example provides a preparation method for a polyester-polyethylene glycol block copolymer, including following steps.

To a 10 mL clean flask equipped with a magnetic stirrer, 6.4 g of PEG with the molecular weight of 6000 was added. The flask was immersed in the oil bath at 130° C. Upon PEG melting, vacuum was applied for about 1.5 h under stirring to remove volatile substances such as residual moisture from the PEG. 0.725 g of D,L-lactide and 2.59 g of L-lactide were added, respectively. After melting, 0.3 g of stannous octoate/L-lactide mixture was added, followed by stirring at 130° C. for 24 h, then cooling to 110° C., and further stirring for 48 h, thereby completing the polymerization reaction, and yielding the block copolymer PDLLA/LLA-PEG-PDLLA/LLA 6000-7.2/28.8/64. In the above, after each addition of materials, brief evacuation, for example, for 30 min, was applied, followed by sealing.

Herein, the preparation of the stannous octoate/L-lactide mixture followed Example 13.

### Example 18

The present example provides a preparation method for a polyester-polyethylene glycol block copolymer, including following steps.

To a 10 mL clean flask equipped with a magnetic stirrer, 6.4 g of PEG with the molecular weight of 6000 was added. The flask was immersed in the oil bath at 130° C. Upon PEG melting, vacuum was applied for about 1.5 h under stirring to remove volatile substances such as residual moisture from the PEG. 3.3 g of L-lactide was added. After melting, 0.3 g of stannous octoate/L-lactide mixture was added, followed by stirring at 130° C. for 24 h, then cooling to 110° C., and further stirring for 48 h, thereby completing the polymerization reaction, and yielding the block copolymer PLLA-PEG-PLLA 6000-36/64. In the above, after each addition of materials, brief evacuation, for example, for 30 min, was applied, followed by sealing.

Herein, the preparation of the stannous octoate/L-lactide mixture followed Example 13.

### Example 19

The present example provides a preparation method for a polyester-polyethylene glycol block copolymer, including following steps.

To a 500 mL clean flask equipped with a magnetic stirrer, 320 g of PEG with the molecular weight of 6000 was added. The flask was immersed in the oil bath at 130° C. Upon PEG melting, vacuum was applied for about 1.5 h under stirring to remove volatile substances such as residual moisture from the PEG. 90 g of D,L-lactide and 80 g of L-lactide were added, respectively. After melting, 10 g of stannous octoate/L-lactide mixture was added, followed by stirring at 130° C. for 24 h, then cooling to 110° C., and further stirring for 48 h, thereby completing the polymerization reaction, and yielding the block copolymer PDLLA/LLA-PEG-PDLLA/LLA 6000-18/18/64. In the above, after each addition of materials, brief evacuation, for example, for 30 min, was applied, followed by sealing.

Herein, the preparation of the stannous octoate/L-lactide mixture followed Example 13.

### Example 20

The present example provides a preparation method for a polyester-polyethylene glycol block copolymer, including following steps.

To a 500 mL clean flask equipped with a magnetic stirrer, 350 g of PEG with the molecular weight of 6000 was added. The flask was immersed in the oil bath at 130° C. Upon PEG melting, vacuum was applied for about 17 h under stirring to remove volatile substances such as residual moisture from the PEG. 150 g of L-lactide was added. After melting, 0.14 g of stannous octoate was added, followed by stirring at 130° C. for 24 h, then cooling to 110° C., and further stirring for 48 h, thereby completing the polymerization reaction, and yielding the block copolymer PLLA-PEG-PLLA 6000-30/70. In the above, after each addition of materials, brief evacuation, for example, for 120 min, was applied, followed by sealing.

### Example 21

The present example provides a preparation method for a polyester-polyethylene glycol block copolymer, including following steps.

To a 500 mL clean flask equipped with a magnetic stirrer, 350 g of PEG with the molecular weight of 6000 was added. The flask was immersed in the oil bath at 130° C. Upon PEG melting, vacuum was applied for about 17 h under stirring to remove volatile substances such as residual moisture from the PEG. 150 g of L-lactide was added. After melting, 0.103 g of stannous octoate was added, followed by stirring at 130° C. for 24 h, then cooling to 110° C., and further stirring for 48 h, thereby completing the polymerization reaction, and yielding the block copolymer PDLA-PEG-PDLA 6000-30/70. In the above, after each addition of materials, brief evacuation, for example, for 120 min, was applied, followed by sealing.

### Example 22

The present example provides a preparation method for a polyester-polyethylene glycol block copolymer, including following steps.

To a 10 mL clean flask equipped with a magnetic stirrer, 5 g of PEG with the molecular weight of 6000 was added. The flask was immersed in the oil bath at 130° C. Upon PEG melting, vacuum was applied for about 1.5 h under stirring to remove volatile substances such as residual moisture from the PEG. 4.7 g of D-lactide was added. After melting, 0.3 g of stannous octoate/D-lactide mixture was added, followed by stirring at 130° C. for 24 h, then cooling to 110° C., and further stirring for 48 h, thereby completing the polymerization reaction, and yielding the block copolymer PDLA-PEG-PDLA 6000-50/50. In the above, after each addition of materials, brief evacuation, for example, for 30 min, was applied, followed by sealing.

Herein, the preparation method for the stannous octoate/D-lactide mixture followed Example 13, except that L-lactide was replaced with an equivalent amount of D-lactide.

### Example 23

The present example provides a preparation method for a polyester-polyethylene glycol block copolymer, including following steps.

To a 10 mL clean flask equipped with a magnetic stirrer, 6.4 g of PEG with the molecular weight of 6000 was added. The flask was immersed in the oil bath at 130° C. Upon PEG melting, vacuum was applied for about 1.5 h under stirring to remove volatile substances such as residual moisture from the PEG. 3.3 g of D-lactide was added. After melting, 0.3 g of stannous octoate/D-lactide mixture was added, followed by stirring at 130° C. for 24 h, then cooling to 110° C., and further stirring for 48 h, thereby completing the polymerization reaction, and yielding the block copolymer PDLA-PEG-PDLA 6000-36/64. In the above, after each addition of materials, brief evacuation, for example, for 30 min, was applied, followed by sealing.

Herein, the preparation method for the stannous octoate/D-lactide mixture followed Example 22.

### Example 24

The present example provides a preparation method for a polyester-polyethylene glycol block copolymer, including following steps.

To a 10 mL clean flask equipped with a magnetic stirrer, 7 g of PEG with the molecular weight of 6000 was added. The flask was immersed in the oil bath at 130° C. Upon PEG melting, vacuum was applied for about 1.5 h under stirring to remove volatile substances such as residual moisture from the PEG. 2.7 g of D-lactide was added. After melting, 0.3 g of stannous octoate/D-lactide mixture was added, followed by stirring at 130° C. for 24 h, then cooling to 110° C., and further stirring for 48 h, thereby completing the polymerization reaction, and yielding the block copolymer PDLA-PEG-PDLA 6000-30/70. In the above, after each addition of materials, brief evacuation, for example, for 30 min, was applied, followed by sealing.

Herein, the preparation method for the stannous octoate/D-lactide mixture followed Example 22.

### Example 25

The present example provides a preparation method for a polyester-polyethylene glycol block copolymer, including following steps.

To a 10 mL clean flask equipped with a magnetic stirrer, 7 g of PEG with the molecular weight of 6000 was added. The flask was immersed in the oil bath at 130° C. Upon PEG melting, vacuum was applied for about 1.5 h under stirring to remove volatile substances such as residual moisture from the PEG. 2.7 g of L-lactide was added. After melting, 0.3 g of stannous octoate/L-lactide mixture was added, followed by stirring at 130° C. for 24 h, then cooling to 110° C., and further stirring for 48 h, thereby completing the polymerization reaction, and yielding the block copolymer PLLA-PEG-PLLA 6000-30/70. In the above, after each addition of materials, brief evacuation, for example, for 30 min, was applied, followed by sealing.

Herein, the preparation method for the stannous octoate/L-lactide mixture followed Example 13.

### Example 26

The present example provides a preparation method for a polyester-polyethylene glycol block copolymer, including following steps.

To a 250 mL clean flask equipped with a magnetic stirrer, 10 g of PEG with the molecular weight of 300 was added. The flask was immersed in the oil bath at 130° C. Vacuum was applied for about 1.5 h under stirring to remove volatile substances such as residual moisture from the PEG. Stannous octoate/lactide mixture was added, followed by stirring at 130° C. for 48 h, then cooling to 110° C., and further stirring for 24 h, thereby completing the polymerization reaction, and yielding the block copolymer PLGA-PEG-PLGA 300-71/19/10. In the above, after each addition of materials, brief evacuation, for example, for 30 min, was applied, followed by sealing.

Herein, the preparation method for the stannous octoate/lactide mixture included: taking 0.15 mL of cyclohexane solution of stannous octoate with concentration of 100 mg/mL, adding 71 g of D,L-lactide and 19 g of glycolide, uniformly mixing, and then performing vacuum evacuation to remove cyclohexane. The entire resultant was added to the above reaction system.

### Example 27

The present example provides a preparation method for a polyester-polyethylene glycol block copolymer, including following steps.

To a 10 mL clean flask equipped with a magnetic stirrer, 5 g of PEG with the molecular weight of 6000 was added. The flask was immersed in the oil bath at 130° C. Upon PEG melting, vacuum was applied for about 2 h under stirring to remove volatile substances such as residual moisture from the PEG. Stannous octoate/L-lactide mixture was added, followed by stirring at 130° C. for 24 h, then cooling to 110° C., and further stirring for 72 h, thereby completing the polymerization reaction, and yielding the block copolymer PLLA-PEG-PLLA 6000-50/50. In the above, after each addition of materials, brief evacuation, for example, for 30 min, was applied, followed by sealing.

Herein, the preparation method for the stannous octoate/L-lactide mixture included: taking 100 μL of cyclohexane solution of stannous octoate with concentration of 10 mg/mL, adding 5 g of L-lactide, uniformly mixing, and then performing vacuum evacuation to remove cyclohexane.

### Example 28

The present example followed the preparation method of Example 27, except that the reaction temperature and duration after adding the stannous octoate/L-lactide mixture were different.

In the present example, after adding the stannous octoate/L-lactide mixture, the resultant was stirred at 130° C. for 48 h, then cooled to 110° C., and further stirred for 72 h, thereby completing the polymerization reaction.

### Example 29

The present example followed the preparation method of Example 27, except that the reaction temperature and duration after adding the stannous octoate/L-lactide mixture were different.

In the present example, after adding the stannous octoate/L-lactide mixture, the resultant was heated to 140° C. and stirred for 24 h, then cooled to 110° C., and further stirred for 72 h, thereby completing the polymerization reaction.

### Example 30

The present example provides a preparation method for a polyester-polyethylene glycol block copolymer, including following steps.

To a 10 mL clean flask equipped with a magnetic stirrer, 5 g of PEG with the molecular weight of 6000 was added. The flask was immersed in the oil bath at 130° C. Upon PEG melting, vacuum was applied for about 2 h under stirring to remove volatile substances such as residual moisture from the PEG. Stannous octoate/L-lactide mixture was added, followed by stirring at 130° C. for 24 h, then cooling to 120° C., and further stirring for 48 h, thereby completing the polymerization reaction, and yielding the block copolymer PLLA-PEG-PLLA 6000-50/50. In the above, after each addition of materials, brief evacuation, for example, for 30 min, was applied, followed by sealing.

Herein, the preparation of the stannous octoate/L-lactide mixture included: taking 300 μL of cyclohexane solution of stannous octoate with concentration of 10 mg/mL, adding 5 g of L-lactide, uniformly mixing, and then performing vacuum evacuation to remove cyclohexane.

### Example 31

The present example followed the preparation method of Example 30, except that the reaction temperature and duration after adding the stannous octoate/L-lactide mixture were different.

In the present example, after adding the stannous octoate/L-lactide mixture, the resultant was stirred at 130° C. for 48 h, then cooled to 110° C., and further stirred for 48 h, thereby completing the polymerization reaction.

### Example 32

The present example followed the preparation method of Example 30, except that the reaction temperature and duration after adding the stannous octoate/L-lactide mixture were different.

In the present example, after adding the stannous octoate/L-lactide mixture, the resultant was heated to 140° C. and stirred for 24 h, then cooled to 110° C., and further stirred for 48 h, thereby completing the polymerization reaction.

### Example 33

The present example provides a preparation method for a micelle formulation, including the following steps.

0.25 g of nimodipine, 12.25 g of the copolymer PDLLA/LLA-PEG-PDLLA/LLA 3350-20/20/60 prepared in Example 5, and 10 mL of ethanol were mixed to obtain a mixed solution, which was fixed to a volume of 25 mL with ethanol, separately charged into glass vials, and stored for a long term after being stoppered and capped. Dilution with DSW yielded the nimodipine polymer micelle formulation.

### Example 34

The present example followed the preparation method for the micelle formulation of Example 33, except that an equivalent mass of the copolymer PDLLA/LLA-PEG-PDLLA/LLA 3350-20/20/60 prepared in Example 12 was used to replace the copolymer of Example 5.

### Comparative Example 1

Comparative Example 1 provides a preparation method for a polyester-polyethylene glycol block copolymer, different from Example 1 in the amount of stannous octoate used.

In Comparative Example 1, the amount of stannous octoate used was 0.048 g.

### Comparative Example 2

Comparative Example 2 provides a preparation method for a polyester-polyethylene glycol block copolymer, different from Example 1 in the amount of stannous octoate used and shortened reaction time.

Comparative Example 2 included: to a 100 mL clean flask equipped with a magnetic stirrer, 30 g of PEG with the molecular weight of 3350 was added. The flask was immersed in the oil bath at 110° C. Upon PEG melting, vacuum was applied for about 16 h under stirring to remove volatile substances such as residual moisture from the PEG. Upon heating to 130° C., 10 g of D,L-lactide and 10 g of L-lactide were added, respectively. After melting, 0.035 g of stannous octoate was added. The polymerization reaction was carried out with stirring at 130° C. for 48 h, yielding the block copolymer PDLLA/LLA-PEG-PDLLA/LLA 3350-20/20/60. In the above, after each addition of materials, brief evacuation, for example, for 30 min, was applied, followed by sealing.

### Experimental Example 1

The polyester-polyethylene glycol block copolymers prepared in different examples and comparative examples were detected for weight-average molecular weight, polydispersity, residual monomer, oligomer content, addition amount of metal tin, and color. The test results are listed in Table 1.

TABLE 1
Test Results of Different Copolymers

| | | | | | | Metal Tin | |
|---|---|---|---|---|---|---|---|
| | | | | Lactide | | Addition | |
| | Copolymer | | | Monomer | Oligomer | Amount | Copolymer |
| No. | Composition | Mw | Mw/Mn | Residue | Content | (ppm) | Color |
| Example 1 | PDLLA/LLA-PEG- | 4889 | 1.100 | 1.54% | 1.38% | 105.2 | light brown |
| | PDLLA/LLA | | | | | | |
| | 3350-20/20/60 | | | | | | |
| Example 2 | PDLLA/LLA-PEG- | 4559 | 1.102 | 1.66% | 2.26% | 122.5 | brown |
| | PDLLA/LLA | | | | | | |
| | 3350-20/20/60 | | | | | | |
| Example 3 | PDLLA/LLA-PEG- | 4290 | 1.131 | 2.37% | 2.59% | 137.7 | white |
| | PDLLA/LLA | | | | | | |
| | 3350-20/20/60 | | | | | | |
| Example 4 | PDLLA/LLA-PEG- | 4473 | 1.110 | 2.09% | 2.71% | 46.9 | dark brown |
| | PDLLA/LLA | | | | | | |
| | 3350-20/20/60 | | | | | | |
| Example 5 | PDLLA/LLA-PEG- | 4776 | 1.112 | 1.68% | 1.67% | 93.5 | coffee brown |
| | PDLLA/LLA | | | | | | |
| | 3350-20/20/60 | | | | | | |
| Example 6 | PLLA-PEG-PLLA | 8567 | 1.118 | 2.20% | 1.45% | 137.7 | brown |
| | 6000-50/50 | | | | | | |
| Example 7 | PLLA-PEG-PLLA | 8400 | 1.106 | 1.66% | 1.68% | 85.0 | pale yellow |
| | 6000-50/50 | | | | | | |
| Example 8 | PDLLA/LLA-PEG- | 4592 | 1.114 | 1.53% | 2.01% | 49.8 | light brown |
| | PDLLA/LLA | | | | | | |
| | 3350-20/20/60 | | | | | | |
| Example 9 | PLLA-PEG-PLLA | 8187 | 1.106 | 2.47% | 2.07% | 117.2 | light brown |
| | 6000-50/50 | | | | | | |
| Example 10 | PLLA-PEG-PLLA | 8381 | 1.106 | 1.65% | 1.87% | 46.9 | pale yellow |
| | 6000-50/50 | | | | | | |
| Example 11 | PDLLA/LLA-PEG- | 4296 | 1.056 | 2.09% | 2.30% | 111.3 | light brown |
| | PDLLA/LLA | | | | | | |
| | 3350-20/20/60 | | | | | | |
| Example 12 | PDLLA/LLA-PEG- | 3995 | 1.083 | 1.79% | 2.90% | 46.9 | light yellow |
| | PDLLA/LLA | | | | | | |
| | 3350-20/20/60 | | | | | | |
| Example 13 | PLLA-PEG-PLLA | 8110 | 1.105 | 1.22% | 0.90% | 87.9 | pale yellow |
| | 6000-43/57 | | | | | | |
| Example 14 | PDLA-PEG-PDLA | 8235 | 1.077 | 1.22% | 1.15% | 87.9 | pale yellow |
| | 6000-43/57 | | | | | | |
| Example 15 | PDLLA/LLA-PEG- | 8126 | 1.179 | 1.29% | 1.67% | 87.9 | yellow |
| | PDLLA/LLA 6000- | | | | | | |
| | 14.3/28.7/57 | | | | | | |
| Example 16 | PDLLA/LLA-PEG- | 8427 | 1.163 | 1.32% | 1.55% | 87.9 | yellow |
| | PDLLA/LLA 6000- | | | | | | |
| | 8.6/34.4/57 | | | | | | |
| Example 17 | PDLLA/LLA-PEG- | 7745 | 1.155 | 1.14% | 1.88% | 87.9 | yellow |
| | PDLLA/LLA 6000- | | | | | | |
| | 7.2/28.8/64 | | | | | | |
| Example 18 | PLLA-PEG-PLLA | 8007 | 1.122 | 1.15% | 1.42% | 87.9 | yellow |
| | 6000-36/64 | | | | | | |
| Example 19 | PDLLA/LLA-PEG- | 7287 | 1.197 | 1.76% | 0.96% | 58.6 | light brown |
| | PDLLA/LLA 6000- | | | | | | |
| | 18/18/64 | | | | | | |
| Example 20 | PLLA-PEG-PLLA | 6919 | 1.121 | 0.98% | 1.87% | 82.0 | light brown |
| | 6000-30/70 | | | | | | |
| Example 21 | PDLA-PEG-PDLA | 6945 | 1.119 | 1.02% | 2.34% | 60.4 | yellow |
| | 6000-30/70 | | | | | | |
| Example 22 | PDLA-PEG-PDLA | 8291 | 1.127 | 1.44% | 1.43% | 87.9 | brown |
| | 6000-50/50 | | | | | | |
| Example 23 | PDLA-PEG-PDLA | 7361 | 1.072 | 1.24% | 2.26% | 87.9 | pale yellow |
| | 6000-36/64 | | | | | | |
| Example 24 | PDLA-PEG-PDLA | 6942 | 1.064 | 1.14% | 2.68% | 87.9 | yellow |
| | 6000-30/70 | | | | | | |
| Example 25 | PLLA-PEG-PLLA | 6855 | 1.063 | 1.13% | 1.96% | 87.9 | pale yellow |
| | 6000-30/70 | | | | | | |
| Example 26 | PLGA-PEG-PLGA | 2794 | 1.517 | 3.00% | 1.96% | 44.0 | pale yellow |
| | 300-71/19/10 | | | | | | transparent |
| Example 27 | PLLA-PEG-PLLA | 8948 | 1.125 | 2.24% | 1.12% | 29.3 | yellow |
| | 6000-50/50 | | | | | | |
| Example 28 | PLLA-PEG-PLLA | 10371 | 1.128 | 1.34% | 1.54% | 29.3 | brown |
| | 6000-50/50 | | | | | | |
| Example 29 | PLLA-PEG-PLLA | 9846 | 1.119 | 2.90% | 2.28% | 29.3 | dark brown |
| | 6000-50/50 | | | | | | |
| Example 30 | PLLA-PEG-PLLA | 7658 | 1.083 | 2.73% | 1.77% | 87.9 | brown |
| | 6000-50/50 | | | | | | |
| Example 31 | PLLA-PEG-PLLA | 8394 | 1.057 | 1.27% | 0.97% | 87.9 | brown |
| | 6000-50/50 | | | | | | |
| Example 32 | PLLA-PEG-PLLA | 9471 | 1.050 | 2.98% | 1.67% | 87.9 | dark brown |
| | 6000-50/50 | | | | | | |
| Comparative | PDLLA/LLA-PEG- | 4907 | 1.121 | 2.02% | 0.79% | 281.3 | light brown |
| Example 1 | PDLLA/LLA 3350- | | | | | | |
| | 20/20/60 | | | | | | |
| Comparative | PDLLA/LLA-PEG- | 4841 | 1.080 | 2.45% | 1.16% | 205.1 | yellow |
| Example 2 | PDLLA/LLA 3350- | | | | | | |
| | 20/20/60 | | | | | | |

Herein, the weight-average molecular weight Mw was determined by GPC. In the GPC test, the chromatographic column was GPC KF-803L (8.0 mm×300 mm, 6 μm), the column temperature was 40° C., the sample injection volume was 20 μL, the detector was AD differential refractometer detector, and the analysis duration was 15 min. The mobile phase was THF. Isocratic elution was performed, at the flow rate of 1 mL/min. The standard was polyethylene glycol.

The polydispersity was ratio of weight-average molecular weight Mw to number-average molecular weight Mn as determined by GPC.

The calculation method for the addition amount of metal tin was: addition amount of metal tin (ppm)=proportion of amount of stannous octoate used×118.71/405.12×10⁶; proportion of amount of stannous octoate used=amount of stannous octoate used/(amount of hydroxyl-containing polyethylene glycol used+amount of cyclic ester monomer used).

The monomer residue was determined and calculated by HPLC, and the specific test method was as follows:
(1) formulating the polyester-polyethylene glycol block copolymer to be tested into an acetonitrile solution of the copolymer with concentration of 30 mg/mL, subjected to filtration via a 0.45 μm nylon filter membrane, and collecting the filtrate as the test solution;
(2) injecting the test solution for detection, recording the chromatogram, substituting the peak area of corresponding monomer in the chromatogram into the standard curve, to calculate the concentration of the monomer in the test solution, and then calculating the content of the monomer in the copolymer sample. Herein, the HPLC detection conditions were as follows:
chromatographic column: Supersil AQ-C18, 5 μm, 4.6 mm×250 mm;
mobile phase A: 0.1 wt % phosphoric acid aqueous solution; and mobile phase B: acetonitrile;
performing gradient elution according to Table 2;
flow rate: 1.0 mL/min;
detection wavelength: 210 nm;
column temperature: 40° C.; and
sample injection amount: 10 μL.

TABLE 2
Gradient Elution Procedure

| Time/min | Mobile Phase A/vol. % | Mobile Phase B/vol. % |
|---|---|---|
| 0 | 98 | 2 |
| 2 | 98 | 2 |
| 25 | 0 | 100 |
| 30 | 0 | 100 |
| 31 | 98 | 2 |
| 41 | 98 | 2 |

The method for acquiring the standard curve included: injecting a series of DLLA standard working solutions (specifically, the solutions being acetonitrile solutions of DLLA, with DLLA concentrations of 1.001 mg/mL, 2.003 mg/mL, 3.004 mg/mL, 5.007 mg/mL, 6.008 mg/mL, 8.011 mg/mL, and 10.014 mg/mL, respectively) separately into the high performance liquid chromatography instrument; under the above detection conditions, measuring corresponding chromatographic peak areas; and plotting the standard curve with the monomer concentration in the series of standard working solutions as the abscissa and the peak area as the ordinate.

The calculation of the oligomer content included: subtracting a peak area of about 13.5 min from the total peak area in the chromatogram as measured by corresponding HPLC method to obtain a peak area value, which was brought into the DLLA standard curve for calculation. ¹H-NMR: in the ¹H-NMR spectrum, the signal peak at δ=5.02-5.08 corresponded to methylene group of lactide, the signal peak at δ=5.11-5.28 corresponded to methylene group in the repeating units of polylactide, integrating the two regions, to calculate the proportion of unreacted lactide, and then calculating the lactide monomer residue amount based on a charging amount of lactide raw material in the formula.

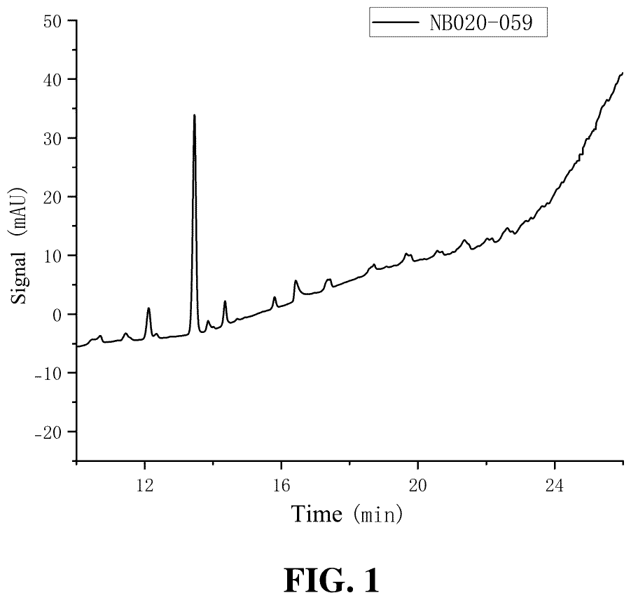

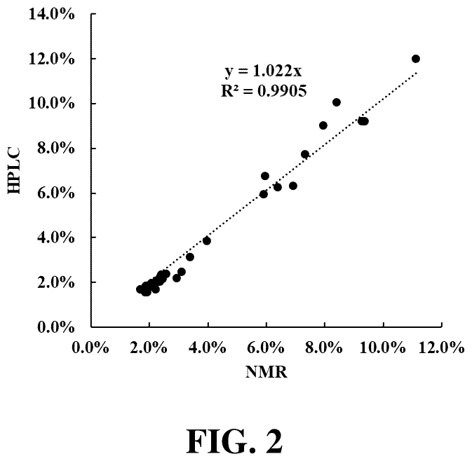

FIG. 1 is an HPLC detection chromatogram of the polyester-polyethylene glycol block copolymer prepared in Example 13 of the present disclosure. As can be seen from the drawing, the main peak with retention duration of about 13.5 min corresponded to lactide, and the remaining minor peaks corresponded to oligomers. In order to validate the accuracy of HPLC for oligomer detection, we adopted a large number of samples (including different raw material ratios, different synthesis conditions, and different batches) to detect the lactide monomer residue results in the polyester-polyethylene glycol block copolymer by both the HPLC detection method and the ¹H-NMR measurement method. Correlation between the detection results of the two methods was analyzed, thereby cross-validating the accuracy of the methods. The test results are shown in FIG. 2, and it can be seen from the drawing that the results of the HPLC detection method and the ¹H-NMR measurement method in the present disclosure were substantially consistent, and the monomer content of the copolymer can be quickly and accurately detected.

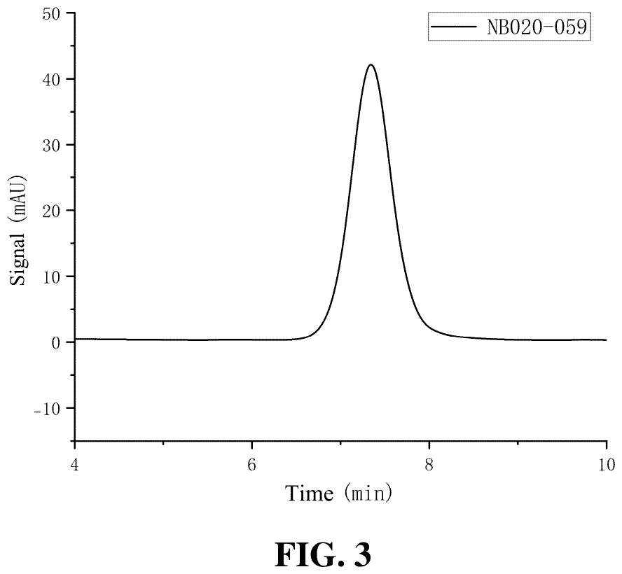

It can be seen from the test results in Table 1 that by employing suitable polymerization reaction conditions, the present disclosure synthesizes the polyester-polyethylene glycol block copolymer meeting requirements of drug carriers for contents of residual metal, monomer, and oligomer in a single step, without purification steps, thus significantly improving the production efficiency and the product yield (theoretically 100%), and the molecular weight distribution of the resulting polymer is favorable. FIG. 3 is a GPC chromatogram of the polyester-polyethylene glycol block copolymer prepared in Example 13. Furthermore, employing the stepwise temperature reduction, compared with isothermal polymerization, not only reduces the monomer residue, but also further decreases the oligomer content, while simultaneously yielding the polymer with a relatively light color.

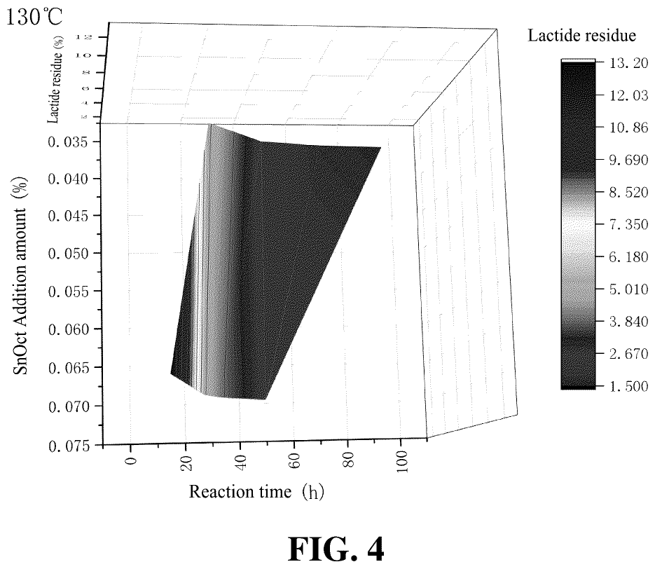

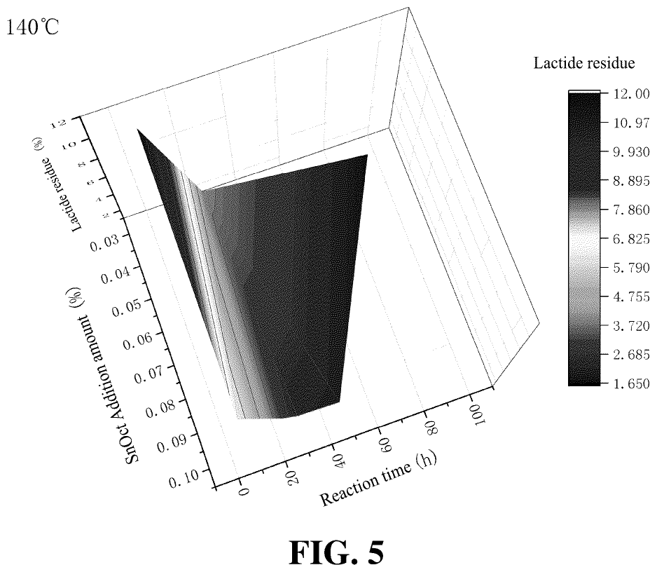

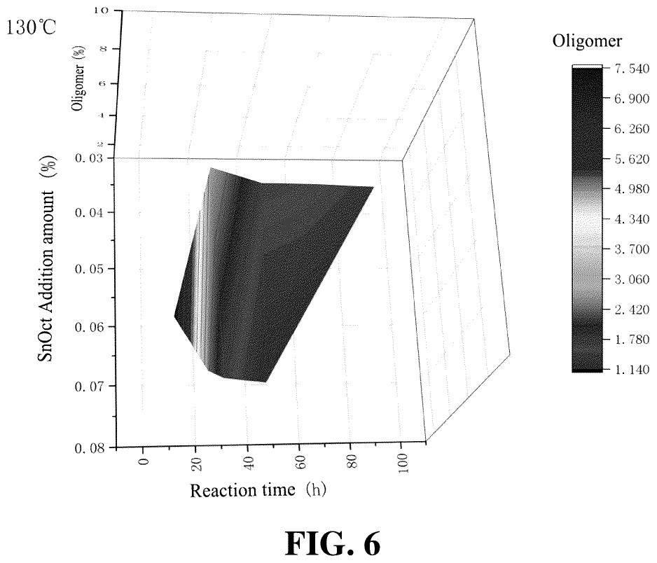

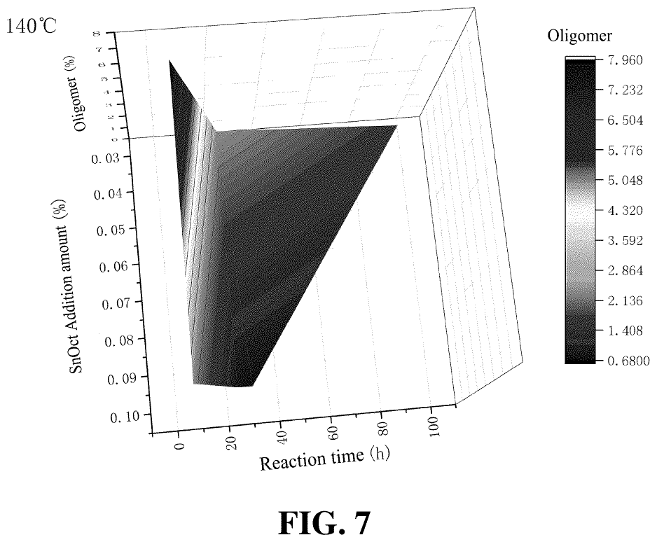

FIG. 4 and FIG. 5 respectively show the influence of catalyst dosage and reaction time on the lactide residue amounts in the polyester-polyethylene glycol block copolymer (PDLLA/LLA-PEG-PDLLA/LLA 3350-20/20/60) prepared under isothermal polymerization at 130° C. and isothermal polymerization at 140° C. in the present disclosure. FIG. 6 and FIG. 7 respectively show the influence of catalyst dosage and reaction time on the oligomer content in the polyester-polyethylene glycol block copolymer (PDLLA/LLA-PEG-PDLLA/LLA 3350-20/20/60) prepared under isothermal polymerization at 130° C. and isothermal polymerization at 140° C. in the present disclosure.

In order to further compare influences of different polymerization temperature conditions on the monomer residue and oligomer content, monomer contents and oligomer contents of reaction systems of Example 1, Example 5, Example 8, Example 11, Example 12, Example 13, Example 14, and Example 19 at different time points were tested, and the test results are listed in Table 3.

TABLE 3
Monomer Content and Oligomer Content at Different
Time Points in Different Reaction Systems

| | | Catalyst | | |
|---|---|---|---|---|
| | Copolymer | Concentration/ | Lactide | Oligomer |
| No. | Composition | Reaction Time | Content | Content |
| Example 1 | PDLLA/LLA-PEG- | 0.036%/27 h | 5.15% | 2.79% |
| | PDLLA/LLA 3350- | 0.036%/49 h | 2.37% | 1.64% |
| | 20/20/60 | 0.036%/72 h | 1.72% | 1.57% |
| | | 0.036%/96 h | 1.54% | 1.38% |
| Example 5 | PDLLA/LLA-PEG- | 0.032%/7 h | 11.98% | 7.96% |
| | PDLLA/LLA 3350- | 0.032%/24 h | 3.89% | 2.36% |
| | 20/20/60 | 0.032%/96 h | 1.68% | 1.67% |
| Example 8 | PDLLA/LLA-PEG- | 0.017%/48 h | 2.27% | 2.46% |
| | PDLLA/LLA 3350- | 0.017%/72 h | 1.83% | 2.53% |
| | 20/20/60 | 0.017%/120 h | 1.45% | 2.12% |
| | | 0.017%/144 h | 1.46% | 2.10% |
| | | 0.017%/150 h | 1.53% | 2.01% |
| Example | PDLLA/LLA-PEG- | 0.038%/24 h | 3.87% | 3.42% |
| 11 | PDLLA/LLA 3350- | 0.038%/48 h | 2.79% | 2.77% |
| | 20/20/60 | 0.038%/72 h | 2.09% | 2.30% |
| Example | PDLLA/LLA-PEG- | 0.016%/24 h | 4.68% | 6.65% |
| 12 | PDLLA/LLA 3350- | 0.016%/48 h | 2.89% | 4.02% |
| | 20/20/60 | 0.016%/72 h | 1.79% | 2.90% |
| Example | PLLA-PEG-PLLA | 0.03%/24 h | 3.44% | 1.77% |
| 13 | 6000-43/57 | 0.03%/48 h | 1.68% | 1.13% |
| | | 0.03%/72 h | 1.22% | 0.90% |
| Example | PDLA-PEG-PDLA | 0.03%/24 h | 2.35% | 1.99% |
| 14 | 6000-43/57 | 0.03%/48 h | 1.45% | 1.33% |
| | | 0.03%/72 h | 1.22% | 1.15% |
| Example | PDLLA/LLA-PEG- | 0.02%/24 h | 3.94% | 1.98% |
| 19 | PDLLA/LLA 6000- | 0.02%/48 h | 2.50% | 1.34% |
| | 18/18/64 | 0.02%/72 h | 1.76% | 0.96% |

It can be seen from the above test results that, by adjusting and controlling the polymerization reaction temperature in the present disclosure, the monomer residue can be decreased, and the oligomer content can be reduced; moreover, the prepared polymer has a relatively light color, and relatively high production efficiency is ensured.

### Experimental Example 2

The formulations prepared in Example 33 and Example 34 were diluted 20 times with D5W. The diluted formulations were tested, and the test results are listed in Table 4.

TABLE 4
Test Results of Formulations

| | | | | Particle | Particle Size |
|---|---|---|---|---|---|
| | | Turbidity | | Size | Dispersity |
| Test Item | Appearance | NTU | pH | (nm) | (PD) |
| Example 33 | yellow | 3.5 | 3.22 | 14.35 | 0.0987 |
| | clear liquid | | | | |
| Example 34 | yellow | 3.0 | 3.12 | 18.85 | 0.0654 |
| | clear liquid | | | | |

It can be seen from the above test results that the polyester-polyethylene glycol block copolymer of the present disclosure can meet the requirements for use as a drug carrier, and can be directly employed for manufacturing formulations such as drug-loaded polymer micelles.

Finally, it should be noted that various examples in the above are merely used for illustrating the technical solutions of the present disclosure, rather than limiting the present disclosure; while the detailed description is made to the present disclosure with reference to the preceding examples, those ordinarily skilled in the art should understand that they still could modify the technical solutions described in various preceding examples, or make equivalent substitutions to some or all of the technical features therein; these modifications or substitutions do not make corresponding technical solutions essentially depart from the scope of the technical solutions of various examples of the present disclosure.

1. A preparation method for a polyester-polyethylene glycol block copolymer, comprising a step of performing a melt ring-opening polymerization reaction on hydroxyl-containing polyethylene glycol and cyclic ester monomer, with stannous octoate as catalyst, wherein
in the melt ring-opening polymerization, an amount of the stannous octoate used ranges from 0.01 wt % to 0.047 wt % of a total amount of the hydroxyl-containing polyethylene glycol and the cyclic ester monomer as raw materials; and
a temperature of the melt ring-opening polymerization reaction ranges from 100° C. to 140° C., and a reaction time ranges from 48 h to 150 h.

2. The preparation method for the polyester-polyethylene glycol block copolymer according to claim 1, wherein the melt ring-opening polymerization reaction comprises: reacting mixed materials at 125-135° C. for 10-24 h, followed by cooling to 105-115° C., and reacting for 24-72 h.

3. The preparation method for the polyester-polyethylene glycol block copolymer according to claim 1, wherein the stannous octoate is added directly or in the form of a premix, wherein the premix comprises:
(a) stannous octoate; and
(b) either hydroxyl-containing polyethylene glycol or cyclic ester monomer.

4. The preparation method for the polyester-polyethylene glycol block copolymer according to claim 3, wherein in the premix, a mass fraction of the stannous octoate ranges from 0.01% to 5%.

5. The preparation method for the polyester-polyethylene glycol block copolymer according to claim 1, wherein the melt ring-opening polymerization reaction is carried out in vacuum or under a protective gas atmosphere.

6. The preparation method for the polyester-polyethylene glycol block copolymer according to claim 1, wherein the hydroxyl-containing polyethylene glycol comprises polyethylene glycol and monomethyl ether polyethylene glycol.

7. The preparation method for the polyester-polyethylene glycol block copolymer according to claim 1, wherein the cyclic ester monomer comprises at least one selected from the group consisting of lactide, glycolide, and caprolactone.

8. The preparation method for the polyester-polyethylene glycol block copolymer according to claim 1, wherein a mass ratio of the cyclic ester monomer to the hydroxyl-containing polyethylene glycol ranges from 0.4:1 to 9:1.

9. A polyester-polyethylene glycol block copolymer, prepared by the preparation method according to claim 1.

10. The preparation method for the polyester-polyethylene glycol block copolymer according to claim 6, wherein the hydroxyl-containing polyethylene glycol has a number-average molecular weight ranging from 200 to 180,000.

11. The preparation method for the polyester-polyethylene glycol block copolymer according to claim 7, wherein the lactide comprises at least one selected from the group consisting of meso-lactide, rac-lactide, D-lactide, and L-lactide.

12. The polyester-polyethylene glycol block copolymer according to claim 1, wherein the polyester-polyethylene glycol block copolymer has a polydispersity Mw/Mn ≤2.0.

13. The polyester-polyethylene glycol block copolymer according to claim 1, wherein the polyester-polyethylene glycol block copolymer has a weight-average molecular weight ranging from 2,000 to 200,000.

14. The polyester-polyethylene glycol block copolymer according to claim 1, wherein the polyester-polyethylene glycol block copolymer has a metal tin content ≤150 ppm.

15. The polyester-polyethylene glycol block copolymer according to claim 14, wherein the polyester-polyethylene glycol block copolymer has a metal tin content ≤60 ppm.

16. The polyester-polyethylene glycol block copolymer according to claim 9, wherein the polyester-polyethylene glycol block copolymer has a residual monomer content ≤3.0 wt %.

17. The polyester-polyethylene glycol block copolymer according to claim 16, wherein the polyester-polyethylene glycol block copolymer has a residual monomer content ≤2.0 wt %.

18. The polyester-polyethylene glycol block copolymer according to claim 9, wherein the polyester-polyethylene glycol block copolymer has an oligomer content ≤3.0 wt %.

19. The polyester-polyethylene glycol block copolymer according to claim 18, wherein the polyester-polyethylene glycol block copolymer has an oligomer content ≤2.0 wt %.
